(19) United States
(12) Patent Application Publication
(10) Pub. No.: US 2009/0252686 A1
(43) Pub. Date: Oct. 8, 2009

(54) Ex Vivo Hyperpolarization of Imaging Agents

(75) Inventor: Charles M. Marcus, Winchester, MA (US)

Correspondence Address: CHOATE, HALL & STEWART/HARVARD UNIVERSITY, TWO INTERNATIONAL PLACE, BOSTON, MA, 02110, US

(73) Assignee: PRESIDENT AND FELLOWS OF HARVARD COLLEGE, Cambridge, MA (US)

(21) Appl. No.: 12/160,327

(22) PCT Filed: Jan. 11, 2007
(86) PCT No.: PCT/US07/00788

§ 371 Date: Oct. 17, 2008

Related U.S. Application Data
(60) Provisional application No. 60/758,245, filed on Jan. 11, 2006.
(60) Provisional application No. 60/783,201, filed on Mar. 16, 2006.
(60) Provisional application No. 60/783,202, filed on Mar. 16, 2006.

(51) Int. Cl. A61K 49/06; A61P 43/00

(52) U.S. Cl. 424/9.3

(57) ABSTRACT

The present invention generally relates to methods for accelerating the ex vivo induction of nuclear hyperpolarization in imaging agents.

### PRIORITY INFORMATION

This application claims priority to U.S. Ser. No. 60/758,245 filed Jan. 11, 2006. This application also claims priority to U.S. Ser. No. 60/783,201 filed Mar. 16, 2006. This application also claims priority to U.S. Ser. No. 60/783,202 filed Mar. 16, 2006. The entire contents of these applications are hereby incorporated by reference.

### BACKGROUND OF THE INVENTION

Magnetic resonance imaging (MRI) systems generally provide for diagnostic imaging of regions within a subject by detecting the precession of the magnetic moments of atomic nuclei in an applied external magnetic field. Spatial selectivity, allowing imaging, is achieved by matching the frequency of an applied radio-frequency (rf) oscillating field to the precession frequency of the nuclei in a quasi-static field. By introducing controlled gradients in the quasi-static applied field, specific slices of the subject can be selectively brought into resonance. By a variety of methods of controlling these gradients in multiple directions, as well as controlling the pulsed application of the rf resonant fields, three-dimensional images representing various properties of the nuclear precession can be detected, giving information about the density of nuclei, their environment, and their relaxation processes. By appropriate choice of the magnitude of the applied quasi-static field and the rf frequency, different nuclei can be imaged.

Typically, in medical applications of MRI, it is the nuclei of hydrogen atoms, i.e., protons, that are imaged. This is, of course, not the only possibility. Information about the environment surrounding the nuclei of interest can be obtained by monitoring the relaxation process whereby the precessional motion of the nuclei is damped, either by the relaxation of the nuclear moment orientation returning to alignment with the quasi-static field following a tipping pulse (on a time scale T1), or by the dephasing of the precession due to environmental effects that cause more or less rapid precession, relative to the applied rf frequency (on a time scale T2). Conventional MRI contrast agents, such as those based on gadolinium compounds, operate by locally altering the T1 or T2 relaxation processes of protons. Typically, this relies on the magnetic properties of the contrast agent, which alters the local magnetic environment of protons. In this case, when images display either of these relaxation times as a function of position in the subject, the location of the contrast agent shows up in the image, providing diagnostic information. Contrast enhancement has also been achieved by utilizing the Overhauser effect, in which an electron transition in a paramagnetic contrast agent is coupled to the nuclear spin system of the endogenous imaging nuclei (e.g., protons). This so-called Overhauser-enhanced magnetic resonance imaging (OMRI) technique increases the polarization of the imaged nuclei and thereby amplifies the acquired signal.

An alternative approach to MRI imaging is to introduce into the subject an imaging agent, the nuclei of which themselves are imaged by the techniques described above. That is, rather than affecting the local environment of endogenous protons in the body and thereby providing contrast in a proton image, the exogenous imaging agent is itself imaged. Such imaging agents include atomic and molecular substances that have non-zero nuclear spin such as 3He, 129Xe, 31P, 29Si, 13C and others (e.g., see U.S. Patent Application Publication 2004/0171928). The nuclei in these substances may be polarized ex vivo by various methods which orient a significant fraction of the nuclei in the agent. The hyperpolarized substance is then introduced into the body. Once in the body, a strong imaging signal is obtained due to the high degree of polarization of the imaging agent. Also there is only a small background signal from the body, as the imaging agent has a resonant frequency that does not excite protons in the body. For example, U.S. Pat. No. 5,545,396 discloses the use of hyperpolarized noble gases for MRI.

Many proposed imaging agents for hyperpolarized MRI have short spin-lattice relaxation (T1) times, requiring that the material be quickly transferred from the hyperpolarizing apparatus to the body, and imaged very soon after introduction into the body, often on the time scale of tens of seconds. For a number of applications, it is desirable to use an imaging agent with longer T1 times. Compared to gases, solid or liquid materials usually lose their hyperpolarization rapidly. Hyperpolarized substances are, therefore, typically used as gases. For example, U.S. Pat. No. 6,453,188 discloses a method of providing magnetic resonance imaging using a hyperpolarized gas that claims to provide a T1 time of several minutes. Protecting even the hyperpolarized gas from losing its magnetic orientation, however, is also difficult in certain applications. For example, U.S. Patent Application Publication No. 2003/0009126 discloses the use of a specialized container for collecting and transporting 3He and 129Xe gas while minimizing contact induced spin relaxation. U.S. Pat. No. 6,488,910 discloses providing 129Xe gas or 3He gas in microbubbles that are then introduced into the body. The gas is provided in the microbubbles for the purpose of increasing the T1 time of the gas. The spin-lattice relaxation time of such gas, however, is still limited.

There is a need, therefore, for imaging agents that provide greater flexibility in designing relaxation times during nuclear magnetic resonance imaging. In particular, there is a need for hyperpolarizable imaging agents with longer T1 times than those already available. Additionally, there is a need for accompanying methods that enable these imaging agents to be hyperpolarized prior to administration.

### SUMMARY OF THE INVENTION

The present invention generally relates to methods for accelerating the ex vivo induction of nuclear hyperpolarization in imaging agents. The imaging agents are solid-state materials that include both non-zero spin nuclei and zero-spin nuclei. The solid imaging agents exhibit longer T1 times than prior art imaging agents (e.g., on the order of hours). Longer T1 times result in prolonged nuclear hyperpolarization with various advantages for MRI applications. However, longer T1 times also lengthen the time required to induce nuclear hyperpolarization. The methods of the present invention shorten the induction process by temporarily shortening the T1 time of the solid imaging agent during the hyperpolarization step. The temporary reduction in the T1 time is achieved using radiation that temporarily increases the concentration of mobile charge carriers (i.e., electrons or holes) within the imaging agent. The temporary presence of strong electron-nuclear dipolar couplings between the mobile charge carriers and the non-zero spin nuclei of the imaging agent reduces the T1 time. Once nuclear hyperpolarization has been induced to a desired level, the long T1 time of the solid imaging agent can be restored by reducing the concentration of mobile charge carriers. This is achieved by removing the radiation and allowing the mobile charge carriers to dissipate or recombine within the imaging agent.

### BRIEF DESCRIPTION OF THE DRAWING

The invention is described with reference to the figure of the drawing, in which:

FIG. 1 is a graph showing measurements of the T1 time for various silicon materials, including micron-scale powders. As shown, T1 times of greater than 1 hour can be achieved in a variety of materials.

### DESCRIPTION OF CERTAIN EMBODIMENTS OF THE INVENTION

This application refers to published documents including patents, patent applications and articles. Each of these published documents is hereby incorporated by reference.

### Introduction

The present invention generally relates to methods for accelerating the ex vivo induction of nuclear hyperpolarization in imaging agents. As used herein, “ex vivo hyperpolarization” refers to methods in which an imaging agent is hyperpolarized before administration to a subject. These ex vivo methods are to be contrasted with “in situ hyperpolarization” methods that involve hyperpolarizing imaging agents after they have been introduced into a subject. The imaging agents are solid-state materials that include both non-zero spin nuclei and zero-spin nuclei. The solid imaging agents exhibit longer T1 times than prior art imaging agents (e.g., on the order of hours). Longer T1 times result in prolonged nuclear hyperpolarization with various advantages for MRI applications as discussed above. However, longer T1 times also lengthen the time required to induce nuclear hyperpolarization. The methods of the present invention shorten the induction process by temporarily shortening the T1 time of the solid imaging agents during the hyperpolarization step. The temporary reduction in the T1 time is achieved using radiation that temporarily increases the concentration of mobile charge carriers (i.e., electrons or holes) within the imaging agent. The temporary presence of strong electron-nuclear couplings between the mobile charge carriers and the non-zero spin nuclei of the imaging agent reduces the T1 time. Once nuclear hyperpolarization has been induced to a desired level, the long T1 time of the solid imaging agent can be restored by reducing the concentration of mobile charge carriers. This is achieved by removing the radiation and allowing the mobile charge carriers to dissipate or recombine within the imaging agent.

### Imaging Agents

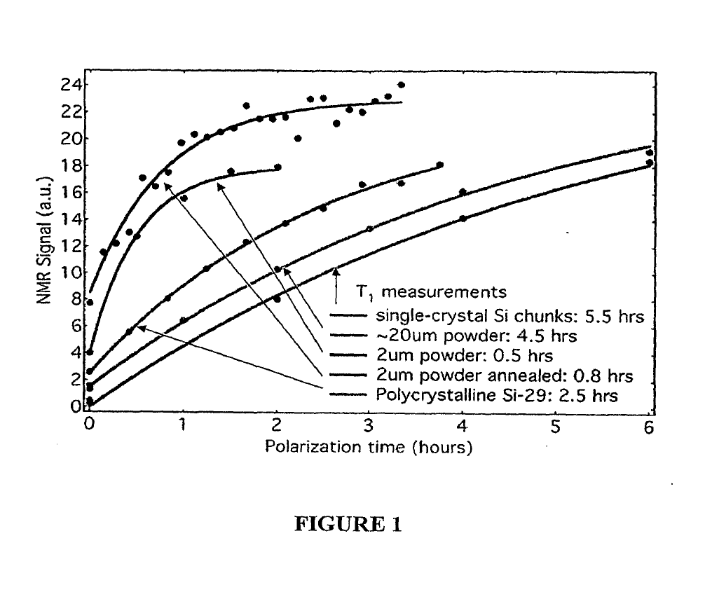

The hyperpolarization methods of the present invention are performed with solid-state imaging agents. Although liquids and solids typically have short relaxation (T1) times, we have discovered that certain solid materials with long T1 times can be manufactured and that these materials can be used as hyperpolarizable imaging agents. For example, FIG. 1 shows measurements of the T1 time for various silicon materials, including micron-scale powders. As shown, T1 times of greater than one hour can be achieved in a variety of materials. It is to be understood that, while the inventive methods enable the preparation and use of materials with long T1 times, the present invention is not limited to such materials. Thus in general, the imaging agents may have T1 times that are shorter than one minute, longer than one minute, longer than ten minutes, longer than thirty minutes, longer than one hour, longer than two hours, or even longer than four hours.

The solid-state imaging agents include both non-zero spin nuclei and zero-spin nuclei (e.g., without limitation, 28Si, 12C, etc.). In certain embodiments, the non-zero spin nuclei are spin-1/2 nuclei (e.g., without limitation, 129Xe, 29Si, 31P, 19F, 15N, 13C, 3He, etc.). However, other non-zero spin nuclei may be used, e.g., without limitation, 10B which is a spin-3 nucleus and/or 11B which is a spin-3/2 nucleus. The solid material can include a mixture of different non-zero spin nuclei. The solid material can also include a mixture of different zero-spin nuclei.

It is to be understood that the relative concentrations of zero-spin and non-zero spin nuclei within the solid material can be tailored by the user. In one embodiment, the concentration of zero-spin nuclei is greater than the concentration of non-zero spin nuclei. For example, the concentration of non-zero spin nuclei can be less than 50%, less than 40%, less than 30%, less than 20%, less than 10%, less than 5%, less than 1% or even less than 0.1% of the total concentration of nuclei in the solid material. In another embodiment, the concentration of non-zero spin nuclei is greater than the concentration of zero-spin nuclei. For example, the concentration of zero spin nuclei can be less than 50%, less than 40%, less than 30%, less than 20%, less than 10%, less than 5%, less than 1% or even less than 0.1% of the total concentration of nuclei in the solid material. In certain embodiments, different isotopes of a particular element can be present at about natural abundance levels. Alternatively, the solid material may be enriched or depleted for a particular isotope. Methods for preparing such materials have been described, e.g., Ager et al., J. Electrochem. Soc. 152:G488, 2005 describes methods for preparing isotopically enriched silicon.

In one aspect, the solid material may include a mixture of an atomic substance that has no nuclear spin and an atomic substance that has a non-zero nuclear spin. For example, 2SSi and 12C have no nuclear spin while 129Xe, 29Si, 31P, 19F, 15N, 13C and 3He have spin-1/2 nuclei. In one embodiment, the material includes silicon nuclei with a natural abundance mixture of isotopes 28Si (zero-spin, about 92.2%), 29Si (spin-1/2, about 4.7%) and 30Si (zero-spin, about 3.1%). In another embodiment, the level of 29Si is higher than its natural abundance level, e.g., higher than about 4.7%, 5%, 7%, 10%, 20%, 30%, 40% or even 50%. In yet another embodiment, the level of 29Si is lower than its natural abundance level, e.g., lower than about 4.7%, 4%, 3%, 2%, 1%, 0.5% or even 0.1%. Methods for preparing silicon materials (e.g., silicon or silica) with varying levels of silicon isotopes have been developed for the computer industry and are well known in the art, e.g., see Haller, J. Applied Physics 77:2857, 1995. In another embodiment, the material includes carbon nuclei with a natural abundance mixture of isotopes 12C (zero-spin, about 98.9%) and 13C (spin-1/2, about 1.1%). In another embodiment, the level of 13C is higher than its natural abundance level, e.g., higher than about 1.1%, 2%, 5%, 10%, 20%, 30%, 40% or even 50%. In yet another embodiment, the level of 13C is lower than its natural abundance level, e.g., lower than about 1.1%, 1%, 0.8%, 0.6%, 0.4%, 0.2% or even 0.1%. Methods for preparing carbon materials with varying levels of carbon isotopes are also known in the art, e.g., see Graebner et al., Applied Physics Letters, 64:2549, 1994.

In general, the inventive material may include any combination of non-zero spin nuclei and zero-spin nuclei. Taking 29Si and 13C as exemplary non-zero spin nuclei, the invention encompasses imaging agents comprising the following exemplary combinations of nuclei and material: 29Si in a silicon (Si) material (e.g., natural abundance silicon, 29Si enriched silicon or 29Si depleted silicon); 29Si in a silica (SiO₂) material (e.g., natural abundance silica, 29Si enriched silica or 29Si depleted silica): 29Si and/or 13C in a silicon carbide (SiC) material; 13C in a carbon material (e.g., diamond or fullerene); 31P in a silicon (Si) material (e.g., phosphorous doped silicon); 10B or 11B in a silicon (Si) material (e.g., boron doped silicon); etc. In one embodiment, the inventive material includes endohedral fullerenes that incorporate non-zero spin nuclei. For example, an inventive material can include a 15N@60C, 15N@80C, etc. endohedral fullerene (where the 15N@ sign indicates an endohedral fullerene with a core 15N nucleus). 15N is not only a spin-1/2 nucleus, but it also has a free spin which facilitates the hyperpolarization methods of the present invention. 129Xe and 3He are other exemplary nuclei that can be incorporated within an endohedral fullerene. These endohedral fullerenes can be prepared based on methods in the art, e.g., Fatouros et al., Radiology 240:756, 2006 which describes methods for preparing endohedral metallofullerene particles.

In one aspect, the imaging agent may include mobile charge carriers that are not generated by irradiation. These “stable” carriers may persist within the imaging agent after irradiation. In one embodiment, these “stable” mobile charge carriers are provided by doping an inventive imaging agent with either n-type or p-type impurities. The presence of these dopants will shorten the T1 time of the imaging agent; by controlling the doping level, the degree of reduction of T1 can be controlled. For example, the T1 times of 29Si in pure silicon doped with various levels of n-type or p-type impurities was investigated in Shulman and Wyluda, Phys. Rev. 103:1127, 1956. The T1 times of 29Si ranged from hours to minutes when the mobile charge carrier concentration was adjusted from 1×10¹⁴ to 1×10¹⁹. N-type impurities had the greater impact on T1 times. It will be appreciated that any impurity type or level can be used. When selecting a particular level of impurity, the user will need to consider the impact on the T1 time. Some applications will favor long T1 times and thus lower impurity levels. Other applications will be less sensitive to T1 and will therefore tolerate higher impurity levels. Precise concentrations of dopants in the inventive solid materials of the invention are readily available commercially (e.g., from Virginia Semiconductor of Fredericksburg, Va.) or can be made using methods known in the semiconductor art (e.g., see Haller, J. Applied Physics 77:2857, 1995).

Exemplary and non-limiting materials that can be used as imaging agents in this aspect of the invention include P- or B-doped silicon. In either case, 29Si nuclei can be hyperpolarized and imaged. P-doped silicon provides both mobile charge carriers and non-zero spin 31P nuclei (spin-1/2). In certain embodiments, the 31P nuclei can be hyperpolarized and used for imaging. Boron has two stable isotopes, 10B (spin-3, 20% natural abundance) and 11B (spin-3/2, 80% natural abundance) which may also be hyperpolarized and imaged. 11B has the advantage of a high NMR receptivity (thus a higher signal for the same polarization density), which may offset the disadvantages of working with a spin higher than ½.

As noted above, the presence of mobile charge carriers within the inventive materials of this aspect of the invention will reduce T1 times as a result of their strong electron-nuclear dipolar couplings with the non-zero spin nuclei. As a result, the weaker inter-nuclear dipolar couplings (e.g., between 29Si nuclei) will have less of an effect on T1. In such embodiments, the level of zero-spin nuclei in the material may have little impact on T1 times and imaging agents with higher concentrations of non-zero spin nuclei (e.g., 29Si or 13C) may be advantageously used in order to generate maximum signal strength. For example, in a P- or B-doped silicon material, the combined concentration of 28Si and 30Si could be less than 50%, less than 40%, less than 30%, less than 20%, less than 10%, less than 5%, less than 1% or even less than 0.1% of the total concentration of nuclei in the material.

The solid imaging agent can be in any form. In certain embodiments, the imaging agent can be in dry particulate form. For example, the imaging agent can be in the form of a powder that includes particles with dimensions in the range of 10 nm to 10 μm. In certain embodiments, the particles may have dimensions in the range of 10 nm to 1 μm. In other embodiments, the particles may have dimensions in the range of 10 to 100 nm. It will be appreciated that in certain embodiments, the particles may be combined and compressed for purposes of administration (e.g., in the form of a tablet) and can be formulated along with other ingredients including pharmaceutically acceptable carriers (e.g., binders, lubricants, fillers, etc.). Alternatively, the imaging agent may be in the form of a suspension with particles having the same range of dimensions. The liquid of the suspension may be aqueous or non-aqueous and may include ingredients that stabilize the suspension (e.g., surfactants) as well as pharmaceutically acceptable carriers. As used herein, the term “pharmaceutically acceptable carrier” means a non-toxic, inert solid, semi-solid or liquid filler, diluting agent, encapsulating material or formulation auxiliary of any type. Remington's Pharmaceutical Sciences Ed. by Gennaro, Mack Publishing, Easton, Pa., 1995 discloses various carriers used in formulating pharmaceutical compositions and known techniques for preparing them. Coloring agents, coating agents, sweetening, flavoring and perfuming agents and preservatives can also be included with an inventive imaging agent. In general, if a carrier is used, it will be selected based on one or more of the route of administration, the location of the target tissue, the imaging agent being delivered, the time course of delivery of the imaging agent, etc.

### Hyperpolarization Methods

Generally, the hyperpolarization methods of the present invention involve irradiating an inventive solid-state imaging agent with a first form or radiation that generates mobile charge carriers (i.e., electrons or holes) within the solid imaging agent and hyperpolarizing at least a portion of the non-zero spin nuclei while at least some of the generated mobile carriers in the step of irradiating are present within the solid imaging agent.

The irradiating and hyperpolarization steps will generally overlap but can be combined in various ways. Thus, in certain embodiments, the step of irradiating and the step of hyperpolarizing may begin and/or end at the same time. In other embodiments, the step of irradiating and the step of hyperpolarizing may begin and/or end at different times. For example, it may prove advantageous to begin the irradiation step before the hyperpolarization step in order to build up a sufficient concentration of carriers before commencing hyperpolarization. In certain embodiments, the irradiation step may end before the hyperpolarizing step ends. This configuration allows the induced carriers to recombine or otherwise leave the region of the target non-zero spin nuclei before the hyperpolarization step has been completed thereby ensuring that the long T1 time of the solid imaging agent has been restored before the end of hyperpolarization.

In general, any form of radiation that can generate mobile charge carriers within the solid imaging agent may be used. The radiation used to generate carriers will depend on the nature of the solid imaging agent. If the solid imaging agent has an electronic band gap then any radiation with an energy greater than the band gap can generate mobile carriers in the form of electron-hole pairs. For example, if the solid imaging agent comprises silicon then any radiation with an energy that is greater than the silicon band gap (about 1.2 eV) could be used. In certain embodiments, radiation with an energy that is greater than about 1.4 eV, 1.6 eV, 1.8 eV or even 2.0 eV could be used. Since the methods of the present invention involve ex vivo hyperpolarization there are no additional restrictions on the type of radiation that can be used (as opposed to in situ methods that can only be performed with radiation that can penetrate the subject). The source of the first form of radiation is equally broad. For example, ambient light may be sufficient for a given solid imaging agent. In other embodiments, a white light, incandescent light, LED light, or laser light source might be used.

Once irradiation begins, the nuclear T1 time of the solid imaging agent will rapidly decrease. The specific steady-state T1 time will depend in part on the energy and intensity of the radiation. Higher intensity radiation will generate more mobile charge carriers and will therefore generally lead to shorter T1 times. Without limitation, the T1 time during irradiation (T1_{with}) may range anywhere from a few microseconds or less to several minutes. These T1 times can be considerably shorter than the T1 times of the solid imaging agents without irradiation (T1_{without}). The irradiation step may last until the desired level of hyperpolarization has been reached. Advantageously, this will generally be a period of time that is shorter than T1_{without}. Typically, the irradiation step will last for at least T1_{with}. Without limitation, in one embodiment, the irradiation step may only need to last for a period of time that is shorter than 10×T1_{with}. In other embodiments, the irradiation step may only need to last for a period of time that is shorter than 5×T1_{with} or 3×T1_{with}.

As previously noted, the irradiation step will generally overlap with a step of hyperpolarizing the solid imaging agent. In general, the hyperpolarizing step will involve placing the solid material within an applied magnetic field. Any magnetic field strength can be employed.

In one embodiment, nuclear hyperpolarization can be generated by “brute force” by placing the imaging agent within a strong applied magnetic field at a temperature close to absolute zero (e.g., see Golman et al., British Journal of Radiology 76:S118, 2003). For example, one could use an applied magnetic field of about 10 T and a temperature of about 10 K or less. More generally, an applied magnetic field of more than 4 T, more than 6 T, more than 8 T or more than 10 T may be used. Similarly, the temperature may be less than 20 K, less than 10K, or less than 5 K.

In another embodiment, the step of hyperpolarizing will include a step of irradiating the solid imaging agent with a second form of radiation that excites electronic spin transitions in the mobile carriers present within the solid imaging agent. The mobile carriers may be those provided by a dopant and/or those provided by the temporary irradiation with the first form of radiation. In certain embodiments, the radiation has a frequency fᵢ within a range of fₑ±fₙ, where fₑ is the Larmor frequency of the mobile carrier and fₙ is the Larmor frequency of the non-zero spin nuclei. This frequency will vary depending on the strength of applied magnetic field which could range from a few mT (e.g., less than 1 T, less than 100 mT, less than 10 mT) to several T (e.g., more than 1 T, more than 2 T, more than 4 T, more than 6 T, more than 8 T or more than 10 T). Depending on the exact frequency of the radiation within the range of fₑ±fₙ, the linewidth of the ESR (electron spin resonance) spectrum of the mobile carriers, and the electron-nuclear dipolar couplings involved, the electronic polarization generated by the radiation will be transferred to the non-zero spin nuclei by one or more of the DNP (dynamic nuclear polarization) mechanisms (i.e., the Overhauser effect, the solid effect and/or thermal mixing).

In general, the imaging agent may be administered to a subject after hyperpolarization using any known route of administration. In one set of embodiments, the subject is an animal, e.g., a mammal. Exemplary mammals include humans, rats, mice, guinea pigs, hamsters, cats, dogs, primates, and rabbits. The imaging agent may be administered orally in the form of a powder, tablet, capsule, suspension, etc. The imaging agent may also be administered by inhalation in the form of a powder or spray. Alternatively, a suspension of the imaging agent may be injected (e.g., intravenously, subcutaneously, intramuscularly, intraperitonealy, etc.) into a tissue or directly into the circulation. Rectal, vaginal, and topical (as by powders, creams, ointments, or drops) administrations are also encompassed.

In certain embodiments, the administered imaging agent is given a sufficient period of time to reach a particular location within the subject prior to detection. In one set of embodiments, the imaging agent is present within an internal cavity of the subject at the time of detection. This could be a gastrointestinal space (e.g., gut, small intestine, large intestine, etc.) or an airway of the subject. In other embodiments, the imaging agent is present within the circulation of the subject at the time of detection. In yet other embodiments, the imaging agent is present within a tissue of the subject at the time of detection.

In certain embodiments, the particles of solid material may be modified to include targeting agents that will direct them to a particular cell type (e.g., a tumor cell) or tissue type (e.g., nerve tissue expressing a particular cell-surface receptor). These modified imaging agents will concentrate in regions of the subject that include the cell or tissue type of interest. Proper targeting of these modified imaging agents may require several minutes or hours post-administration to allow for efficient concentration at the site of interest. Solid imaging agents with long T1 times are therefore particularly advantageous for these applications.

The targeting agents can be associated with particles by covalent or non-covalent bonds (e.g., ligand/receptor type interactions). In one embodiment, patterning of surfaces can be used to promote non-covalent bonds between the targeting agent and inventive particles. Alternatively, a whole host of synthetic methods exist for chemically functionalizing the surfaces of inventive particles to produce surface moieties that form covalent or non-covalent bonds with targeting agents. For example, Bhushan et al., Acta Biomater. 1:327, 2005 describes both chemical conjugation and surface patterning methods for associating biomolecules with silicon particle surfaces. Shirahata et al., Chem. Rec. 5:145, 2005 describes the chemical modification of a silicon surface using monolayers and methods for associating biomolecules with these layers. Nakamura et al., Acc. Chem. Res. 36:807, 2003; Pantarotto et al., Mini Rev. Med. Chem. 4:805, 2004; and Katz et al., Chemphyschem 5:1084, 2004 provide reviews of methods for functionalizing carbon fullerenes and thereby associating them with biomolecules.

It is also to be understood that any ligand/receptor pair with a sufficient stability and specificity may be employed to associate a targeting agent with a particle. In general, the ligand/receptor interaction should be sufficiently stable to prevent premature release of the targeting agent. To give but one example, a targeting agent may be covalently linked with biotin and the particle surface chemically modified with avidin. The strong binding of biotin to avidin then allows for association of the targeting agent and particle. Ahmed et al., Biomed. Microdevices 3:89, 2004 describe this approach for silicon particles. Capaccio et al., Bioconjug. Chem. 16:241, 2005 describe this approach for carbon fullerenes. In general, possible ligand/receptor pairs include antibody/antigen, protein/co-factor and enzyme/substrate pairs. Besides biotin/avidin, these include for example, biotin/streptavidin, FK506/FK506-binding protein (FKBP), rapamycin/FKBP, cyclophilin/cyclosporin and glutathione/glutathione transferase pairs. Other suitable ligand/receptor pairs would be recognized by those skilled in the art.

A variety of suitable targeting agents are known in the art (e.g., see Cotten et al., Methods Enzym. 217:618, 1993; Garnett, Adv. Drug Deliv. Rev. 53:171, 2001). For example, any of a number of different agents which bind to antigens on the surfaces of target cells may be employed. Antibodies to target cell surface antigens will generally exhibit the necessary specificity for the target antigen. In addition to antibodies, suitable immunoreactive fragments may also be employed, such as the Fab, Fab′, or F(ab′)₂ fragments. Many antibody fragments suitable for use in forming the targeting agent are already available in the art. Similarly, ligands for any receptors on the surface of the target cells may suitably be employed as a targeting agent. These include any small molecule or biomolecule (including peptides, lipids and saccharides), natural or synthetic, which binds specifically to a receptor (e.g., a protein or glycoprotein) found at the surface of the desired target cell.

Once the imaging agent has been administered to a subject, the hyperpolarized nuclei within the imaging agent can now be detected using appropriate radiation to excite spin transitions of the non-zero spin nuclei. This detection step can be performed at any field strength. Optionally, the nuclear spin signals can also be used to image the spatial distribution of the imaging agent using any known MRI technique, e.g., see MRI in Practice Ed. by Westerbrook et al., Blackwell Publishing, Oxford, UK, 2005. Signal acquisition can be repeated for as long as the imaging agent is present within the subject and retains its nuclear hyperpolarization. In certain embodiments, the imaging agent can be detected and optionally imaged at different points in time.

### Other Embodiments

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

We claim:

1. A method comprising steps of:
providing a solid imaging agent that includes non-zero spin nuclei and zero-spin nuclei;
irradiating the solid imaging agent with a first form of radiation that generates mobile charge carriers within the solid imaging agent; and
hyperpolarizing at least a portion of the non-zero spin nuclei while at least some of the mobile charge carriers generated in the step of irradiating are present within the solid imaging agent.

2. The method of claim 1, wherein the solid imaging agent includes non-zero spin nuclei selected from the group consisting of 129Xe, 29Si, 31P, 19F, 15N, 13C, 1B, and 10B.

3. The method of claim 1, wherein the solid imaging agent includes 29Si nuclei.

4. The method of claim 1, wherein the solid imaging agent includes 13C nuclei.

5. The method of claim 3, wherein the solid imaging agent includes 28Si nuclei.

6. The method of claim 3, wherein the solid imaging agent includes 12C nuclei.

7. The method of claim 4, wherein the solid imaging agent includes 28Si nuclei.

8. The method of claim 4, wherein the solid imaging agent includes 12C nuclei.

9. The method of claim 3, wherein the 29Si nuclei are present at natural abundance levels.

10. The method of claim 3, wherein the 29Si nuclei are present at lower than natural abundance levels.

11. The method of claim 3, wherein the 29Si nuclei are present at higher than natural abundance levels.

12. The method of claim 1, wherein the solid imaging agent includes 29Si nuclei in a silicon material.

13. The method of claim 1, wherein the solid imaging agent includes 29Si nuclei in a silica material.

14. The method of claim 1, wherein the solid imaging agent includes 29Si and/or 13C nuclei in a silicon carbide material.

15. The method of claim 1, wherein the solid imaging agent includes 13C nuclei in a carbon material.

16. The method of claim 1, wherein the solid imaging agent includes 31P nuclei in a silicon material.

17. The method of claim 1, wherein the solid imaging agent includes 10B and/or 11B nuclei in a silicon material.

18. The method of claim 1, wherein the solid imaging agent includes 15N nuclei in a carbon material.

19. The method of claim 18, wherein the carbon material is an endohedral fullerene.

20. The method of claim 1, wherein the step of irradiating and the step of hyperpolarizing begin at the same time.

21. The method of claim 1, wherein the step of irradiating and the step of hyperpolarizing end at the same time.

22. The method of claim 1, wherein the step of irradiating and the step of hyperpolarizing begin and end at the same time.

23. The method of claim 1, wherein the step of irradiating and the step of hyperpolarizing begin at different times.

24. The method of claim 1, wherein the step of irradiating and the step of hyperpolarizing end at different times.

25. The method of claim 1, wherein the step of irradiating and the step of hyperpolarizing begin and end at different times.

26. The method of claim 1, wherein the step of irradiating begins before the step of hyperpolarizing begins.

27. The method of claim 1, wherein the step of irradiating ends before the step of hyperpolarizing ends.

28. The method of claim 1, wherein the solid imaging agent has an electronic band gap and the first form of radiation has an energy greater than the electronic band gap.

29. The method of claim 1, wherein the solid imaging agent comprises silicon.

30. The method of claim 29, wherein the first form of radiation has an energy that is greater than about 1.2 eV.

31. The method of claim 29, wherein the first form of radiation has an energy that is greater than about 1.4 eV.

32. The method of claim 29, wherein the first form of radiation has an energy that is greater than about 1.6 eV.

33. The method of claim 29, wherein the first form of radiation has an energy that is greater than about 1.8 eV.

34. The method of claim 29, wherein the first form of radiation has an energy that is greater than about 2.0 eV.

35. The method of claim 1, wherein the T1 time of the non-zero spin nuclei without the first form of irradiation (T1_{without}) is longer than one hour.

36. The method of claim 1, wherein the T1 time of the non-zero spin nuclei with the first form of irradiation (T1_{with}) is shorter than the T1 time of the non-zero spin nuclei without the first form of irradiation (T1_{without}).

37. The method of claim 1, wherein the step of irradiating lasts for a period of time that is shorter than the T1 time of the non-zero spin nuclei without the first form of irradiation (T1_{without}).

38. The method of claim 1, wherein the step of irradiating lasts for a period of time that is longer than the T1 time of the non-zero spin nuclei with the first form of irradiation (T1_{with}).

39. The method of claim 38, wherein the step of irradiating lasts for a period of time that is shorter than 10×T1_{with}.

40. The method of claim 38, wherein the step of irradiating lasts for a period of time that is shorter than 5×T1_{with}.

41. The method of claim 38, wherein the step of irradiating lasts for a period of time that is shorter than 3×T1_{with}.

42. The method of claim 1, wherein the step of hyperpolarizing comprises a step of:
placing the solid imaging agent within an applied magnetic field.

43. The method of claim 42, wherein the step of hyperpolarizing is performed at a temperature of less than 20 K and the applied magnetic field has a strength of more than 4 T.

44. The method of claim 43, wherein the step of hyperpolarizing is performed at a temperature of less than 10 K and the applied magnetic field has a strength of more than 10 T.

45. The method of claim 42, wherein the step of hyperpolarizing further comprises a step of:
irradiating the solid imaging agent with a second form of radiation that excites electronic spin transitions in mobile charge carriers present within the solid imaging agent.

46. The method of claim 45, wherein the second form of radiation has a frequency fᵢ in the range of fₑ±fₙ, where fₑ is the Larmor frequency of the mobile charge carriers and fₙ is the Larmor frequency of the non-zero spin nuclei.

47. The method of claim 1 further comprising a step of:
administering the solid imaging agent to a subject after the step of hyperpolarizing.

48. The method of claim 47, wherein the solid imaging agent is administered to the subject in the form of particles.

49. The method of claim 48, wherein the particles have dimensions in the range of 10 nm to 10 μm.

50. The method of claim 48, wherein the particles have dimensions in the range of 10 nm to 1 μm.

51. The method of claim 48, wherein the particles have dimensions in the range of 10 nm to 100 nm.

52. The method of claim 47, wherein the solid imaging agent is administered to the subject in the form of a suspension of particles.

53. The method of claim 47, wherein the subject is an animal.

54. The method of claim 47, wherein the subject is a mammal.

55. The method of claim 47, wherein the subject is selected from the group consisting of rats, mice, guinea pigs, hamsters, cats, dogs, primates and rabbits.

56. The method of claim 47, wherein the subject is a human.

57. The method of claim 47, wherein the solid imaging agent is administered orally.

58. The method of claim 47, wherein the solid imaging agent is administered by inhalation.

59. The method of claim 47, wherein the solid imaging agent is administered by injection.

60. The method of claim 47 further comprising a step of:
detecting the hyperpolarized non-zero spin nuclei while the solid imaging agent is present within the subject.

61. The method of claim 60, wherein the spatial distribution of the solid imaging agent within the subject is imaged by magnetic resonance imaging.

62. The method of claim 61, wherein the spatial distribution of the solid imaging agent within the subject is monitored over time.

63. The method of claim 60, wherein the step of detecting is performed after waiting for a sufficient period of time to allow the solid imaging agent to reach a particular location within the subject.

64. The method of claim 60, wherein the solid imaging agent is present within an internal cavity of the subject at the time of detection.

65. The method of claim 60, wherein the solid imaging agent is present within a gastrointestinal space of the subject at the time of detection.

66. The method of claim 60, wherein the solid imaging agent is present within an airway of the subject at the time of detection.

67. The method of claim 60, wherein the solid imaging agent is present within a circulatory system of the subject at the time of detection.

68. The method of claim 60, wherein the solid imaging agent is present within a tissue of the subject at the time of detection.

69. The method of claim 60, wherein the solid imaging agent is associated with a targeting agent that binds with an antigen present on the surface of a cell.

70. The method of claim 69, wherein the targeting agent is an antibody or an immunoreactive fragment of an antibody for the antigen present on the surface of the cell.

71. The method of claim 69, wherein the targeting agent is a ligand and the antigen present on the surface of the cell is a receptor for the ligand.
